(19) United States
(12) Patent Application Publication
(10) Pub. No.: US 2019/0183649 A1
(43) Pub. Date: Jun. 20, 2019

(54) MEDICAL SECURING DEVICE FOR SECURING AN IMPLANT DEVICE WITH A SECURING MEMBER

(71) Applicant: Medtentia International Ltd Oy, Espoo (FI)

(72) Inventors: William ALLEN, Espoo (FI); Allan BACHMAN, Espoo (FI); Adam LEHMAN, Espoo (FI); Jonas TYNNERSTÅL, Espoo (FI); Olli KERÄNEN, Bjärred (SE)

(21) Appl. No.: 16/325,920

(22) PCT Filed: Aug. 16, 2017
(86) PCT No.: PCT/FI2017/050576

§ 371 Date: Feb. 15, 2019

(30) Foreign Application Priority Data
Aug. 16, 2016 (EP) 16184290.1

(51) Int. Cl. A61F 2/24; A61B 17/064; A61B 17/068

(52) U.S. Cl. CPC A61F 2/2466; A61F 2/2445; A61B 17/0644; A61B 17/0686; A61B 2017/00243; A61F 2230/0091; A61F 2220/0016; A61F 2220/0066; A61B 17/0684

(57) ABSTRACT

A medical securing device for securing an implant device, such as a cardiac implant device with a securing member into a tissue, such an annulus of a heart valve comprises a first elongated stem having proximal and distal ends. The distal end of the first stem has a receiving member for receiving the securing member. During a mutual movement of the distal end of the first stem and the receiving member, the distal end of the first stem is configured to introduce with the receiving member the securing member to the implant device to secure the implant device to the tissue with the securing member.

### TECHNICAL FIELD OF THE INVENTION

The invention relates to a medical securing device for securing an implant device with a securing member, and especially a cardiac implant (annuloplasty medical) device. In particularly the invention relates to a medical securing device for an open-heart-operation and for securing the cardiac implant device into an annulus of a heart valve, such as a mitral valve or tricuspid valve, comprised of valve tissue and including the annulus and a plurality of leaflets.

### BACKGROUND OF THE INVENTION

FIG. 1A illustrates a portion of the heart 12, the mitral valve 18, and the left ventricle 14. The mitral valve is at its boundary circumferenced by an annulus 20. The valve has two cusps or leaflets 22, 24. Each of these cusps or leaflets 22, 24 are connected to a respective papillary muscle 27, 29 via their respective connecting chordae 26, 28. In normal healthy individuals the free edges of the opposing leaflets will close the valve by coaptation. However, for some individuals the closure is not complete, which results in a regurgitation, also called valvular insufficiency, i.e. back flow of blood to the left atrium making the heart less effective and with potentially severe consequences for the patient. FIG. 1B illustrates a mitral valve 18, in which the leaflets 22, 24 do not close properly. This commonly occurs when the annulus 20 becomes dilated. One surgical procedure to correct this is to remove a portion of the leaflet 24 and stitch the cut edges together with one another. The procedure will pull back the annulus 20 to a more normal position. However the strength of the leaflet 24 is altered. Similar problems with a less effective heart function occur if one or both leaflets are perforated to such an extent that blood is flowing towards the left atrium, although the leaflets close properly.

In some conditions of degenerated heart function, the leaflets do not present a solid surface, as in a degenerative valve disease. The leaflet may also be ruptured, most commonly at an edge of a leaflet, resulting in an incomplete coaptation. Hence, cardiac devices and methods are developed for repairing of one or more leaflets of a heart valve, or other related anatomical structures, such as the chordae attached to the ventricular side of leaflets.

FIG. 2A-B illustrate prior art cardiac implant devices and method for repairing of one or more leaflets of a heart valve as is described in the applicant's previous EP-patent (EP 1 853 199 B1), where the device 40 comprises a first and a second loop-shaped support 42, 44, which are connected to each other by means of a connecting part 48 so as to form a coil-shape. The coil-shape of the device is advantageous during insertion, since the device 40 may then be rotated into position, as described in the patent in more details. One of the supports 44 may be open, e.g. C or D or any other anatomical shaped such that the support 44 presents an end to lead the movement of the support 44 when being rotated into position. The position of the supports 42, 44 are secured by fasteners 156, which are inserted and fastened by hand or small screwdriver.

It is found that the prior art cardiac implant devices, such as depicted above, work very well, but there are still some disadvantages relating to the securing of the cardiac device into the annulus of the heart valve. The cardiac devices are typically sutured by a needle and yarns, which is time consuming, because in practise it is needed at least seven knots to be tied in order to have even some certainty that the device is secured. In addition, the device is sutured typically by one yarn, which has a drawback namely if one or more knots is/are loosen or the yarn is broken, then the whole securing will come loosen or broken.

The cardiac implant devices are also secured by screws. However, the screws are very small, the assembling, positioning and controlling of which are extremely difficult. The screws must be inserted through the both the first and second (upper and lower) loop-shaped support portions 42, 44 (tiny holes in both of the portions), which is highly demanding, because if the first screw is tightened too much, it will distort the portions little bit and thus misaligning the other holes and thereby making it impossible to inserting the other screws. Furthermore there is a huge risk to drop the small screws into the cardiac structure, because for example any safety blankets cannot be used. In addition also magnetic material cannot be used due to possible later magnetic imaging.

### SUMMARY OF THE INVENTION

It is an object of the invention to alleviate and eliminate the problems relating to the known prior art. Especially the object of the invention is to provide a medical securing device for securing an implant device into a tissue, such as a cardiac implant device with a securing member into an annulus of a heart valve in an easy, fast and safe manner.

The object of the invention can be achieved by the features of independent claims.

The invention relates to a medical securing device for securing an implant device with a securing member into a tissue according to claim 1.

According to an embodiment of the invention a medical securing device for securing an implant device with a securing member into a tissue, such as into an annulus of a heart valve, comprises at least a first elongated stem. The first stem has proximal and distal ends, and the distal end has a receiving member for receiving the securing member. The receiving member is advantageously a piston type member, which is configured to receive the securing member, such as pinch, press or lock the securing member mechanically. In addition the medical securing device is advantageously configured to under manipulation, such as during a movement of the distal end of the first stem and the receiving member in relation to each other, move, introduce and secure, such as press, bend, twist or otherwise secure the securing member to the implant device and thereby to secure the upper and/or lower portion of the implant device to the tissue with the securing member.

According to an embodiment the distal end of the first stem has a hollow structure, such as a conduit or channel, where the receiving member is arranged in a movable manner so that during the movement the receiving member moves and thereby introduces the securing member from the hollow structure to the implant device thereby securing the implant device to the tissue with the securing member.

In addition according to an embodiment the medical securing device may further comprise also a second elongated stem adjacent to the first stem.

The first and second stems are configured to be moved advantageously under the same manipulation or movement in relation to each other in their longitudinal direction, both stems having proximal and distal ends. The distal end of the second stem has a counterpart portion, such as a clamp and/or anvil. The counterpart portion can be used together the distal end of the first stem for clamping the implant device between the counterpart portion and the distal end, but also together the receiving member for stapling the securing member to the implant device and/or to the tissue, such as to the annulus.

Advantageously the first elongated stem is made of one piece of material, as well as especially the second elongated stem is also made of one piece of material, whereupon very strong and stable securing device structure can be achieved. This is very advantageous e.g. when the securing member, such as a stable, is pushed through the tissue and bent by the counterpart portion of the second elongated stem, such as the clamp or anvil, because the dimensions of the annulus and the implant device are small. The unstable device structure, especially the second elongated stem, might bend or twist and thus being unable to guide the securing member correctly through the tissue or guide and bend the securing member around or into the contact with the cardiac implant device and/or to the annulus, when introduced by the first elongated stem and receiving member.

The distal end of the second stem is advantageously configured to be introduced to the opposite portion of the tissue, such as to the valve tissue than the distal end of the first stem or to the lower portion of the implant device, when lower portion is used. In addition the distal end is configured to produce counterforce via the counterpart portion to the distal end of the first stem, for example when the second stem is pulled, and thereby clamping the implant device between the distal ends of the first and second stems when the distal end of the first and/or second stems is/are moved in the longitudinal direction towards the implant device. In addition during the additional movement the distal end of the first stem with the receiving member or the receiving member as such is configured to introduce the securing member to the implant device to secure at least portion of the implant device to the tissue, such as to the annulus of the valve with the securing member.

The securing member may be e.g. a staple the one end of which is bendable at least partially around or into the implant device under the pressing force induced by the distal end of the first stem and/or counterpart portion of the second stem. The securing member may also be e.g. a staple having at least one hook-shaped end to be introduced at least partially around or into the implant device. In addition the securing member may be also a helical clip, locking clip, pointed screw, spring clip, skin staple, pin, or circular clip, as an example. The securing member may comprise for example shape memory material, metal or polymer or any other material with memory function, as an example. In addition the second end or both ends of the securing member may be sharpened (atraumatic) so to help the penetration of the securing member into the annulus tissue.

It is to be noted in connection with the heart valve that the heart valve is for example a mitral valve or tricuspid valve, and comprises valve tissue including the annulus and a plurality of leaflets. In addition it is to be noted that the implant device used in the invention may comprise an upper or lower portion, or both. The portions are advantageously as loops and at least one of them can be open loop, such as C or D shaped portion (or any other suitable anatomical shape), whereby, in use, a portion of the valve tissue is coupled with the upper or lower portion, or trapped between the upper and lower portions of the cardiac implant.

In addition it is to be noted that the medical securing device described in this document can be used for securing different kinds of implant devices to the tissue, but particularly cardiac implant devices to the annulus of the valve. Thus even if the cardiac implant devices are described below and in this document in more details, it is be understood that the same inventive principle of the medical securing device can be applied by securing also other types of implant devices into different tissues, such as tissue or skin transplant beneath the skin or the like.

The present invention offers advantages over the known prior art, such as an easy, safe and time saving manner to reliable securing the cardiac implant device to the annulus of the valve with the securing member. In addition the securing process including both the clamping and positioning of the cardiac implant device, as well as the securing the cardiac implant device in a reliable manner into the right position can be performed in a very natural way and advantageously by one movement. Most advantageously the movement can be just one continuous movement, such as for example by pressing or pulling the operation member(s) in the handle bar of the medical securing device.

The exemplary embodiments presented in this text are not to be interpreted to pose limitations to the applicability of the appended claims. The verb “to comprise” is used in this text as an open limitation that does not exclude the existence of also un-recited features. The features recited in depending claims are mutually freely combinable unless otherwise explicitly stated.

The novel features which are considered as characteristic of the invention are set forth in particular in the appended claims. The invention itself, however, both as to its construction and its method of operation, together with additional objects and advantages thereof, will be best understood from the following description of specific example embodiments when read in connection with the accompanying drawings.

### BRIEF DESCRIPTION OF THE DRAWINGS

Next the invention will be described in greater detail with reference to exemplary embodiments in accordance with the accompanying drawings, in which:

FIGS. 1A-1B illustrate schematically a portion of a heart and mitral valve,

FIGS. 2A-2B illustrate a prior art cardiac implant device for repairing of one or more leaflets of a heart valve, and

FIGS. 3A-13 illustrate examples of medical securing devices according to advantageous embodiments of the invention.

### DETAILED DESCRIPTION

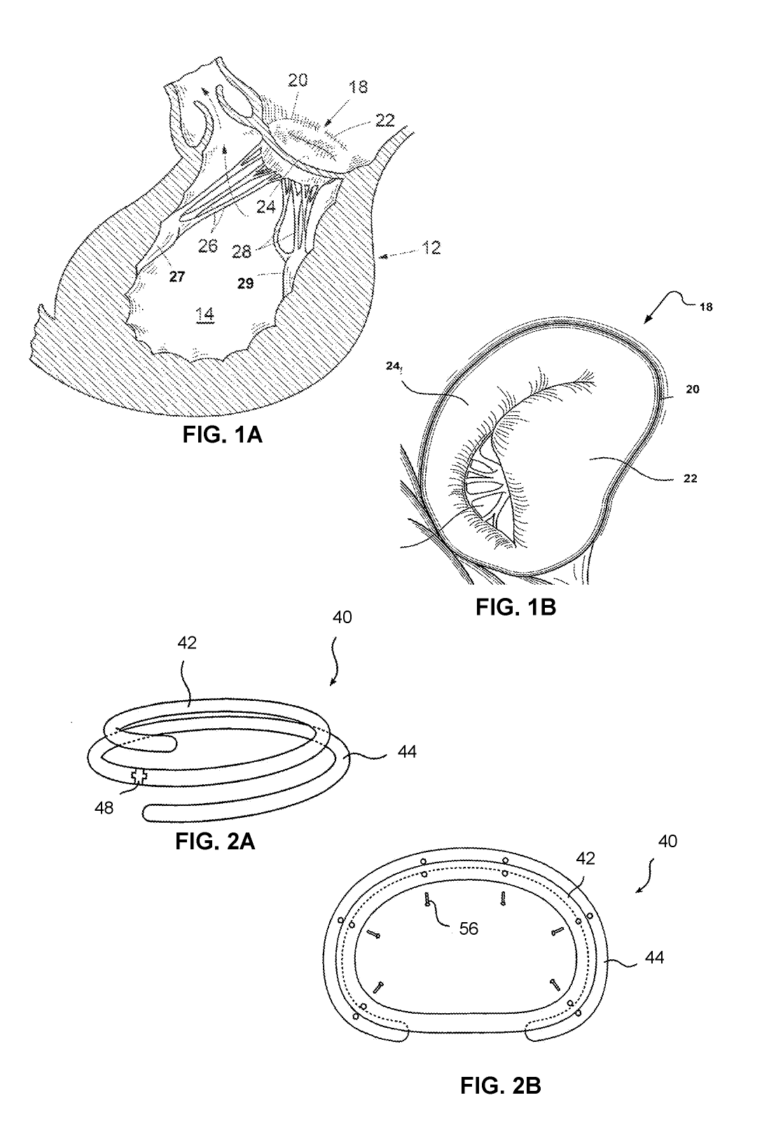

FIGS. 1A-1B and 2A-2B are already discussed in more details in connection with the background of the invention portion above.

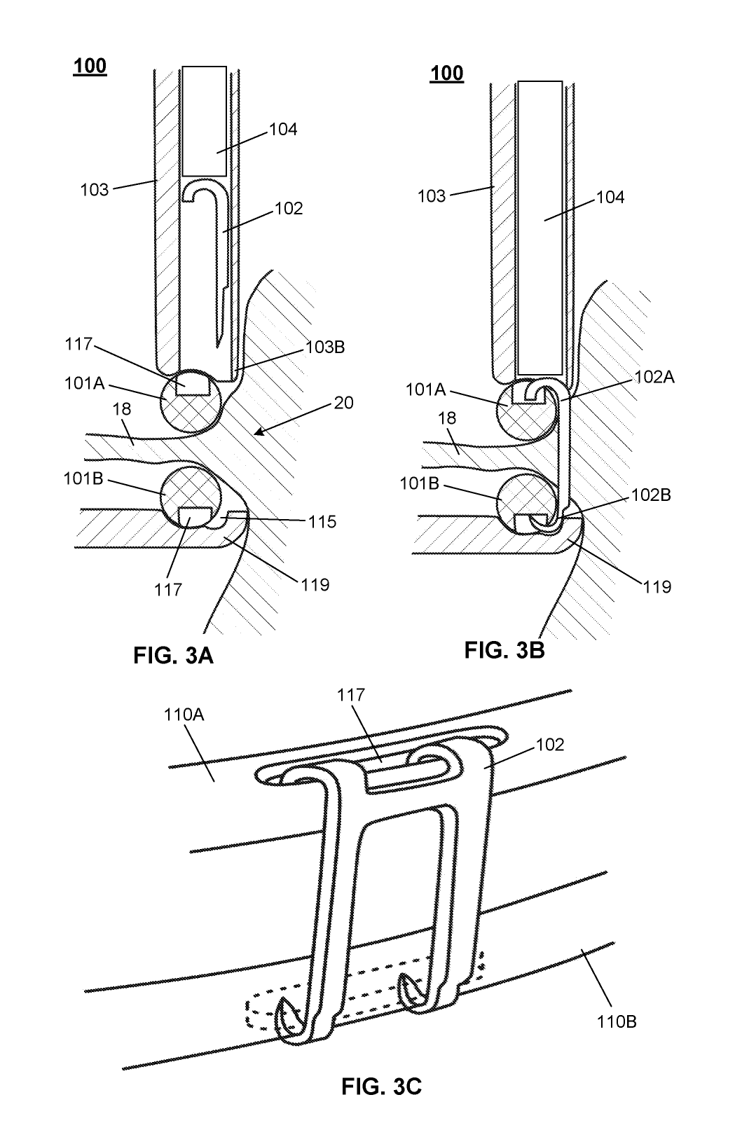

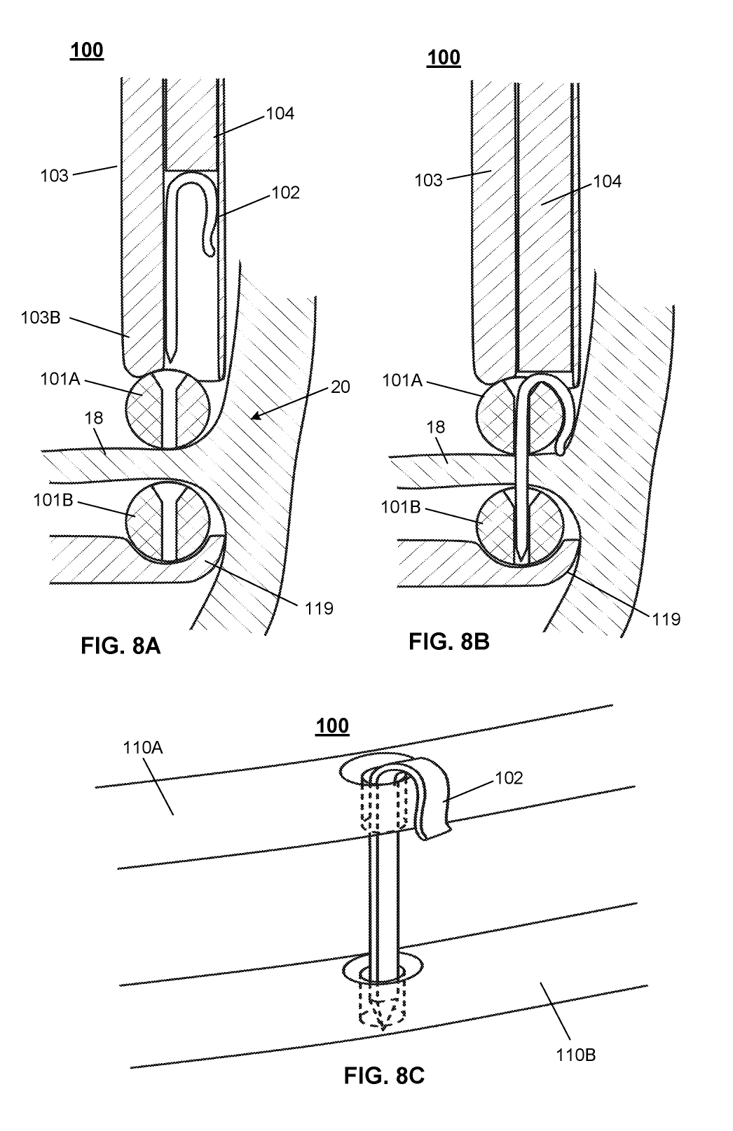

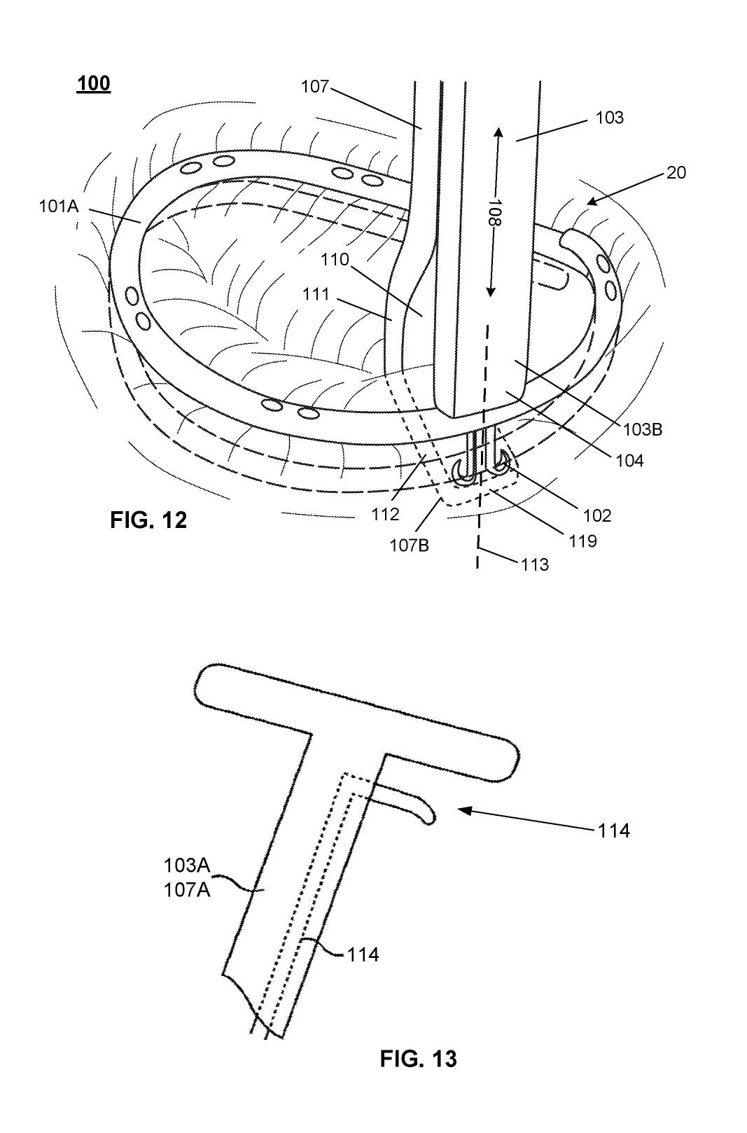

FIGS. 3A-13 illustrate examples of medical securing devices 100 according to advantageous embodiments of the invention, where the medical securing device 100 comprises at least a first elongated stem 103. The first stem has proximal 103A and distal 103B ends (FIG. 12), and the distal end 103B has a receiving member 104 for receiving the securing member 102. The distal end 103B of the first stem and the receiving member 104 can be moved in relation to each other, whereupon they are configured to introduce and secure the securing member 102 to the cardiac implant device 101 and thereby to secure the upper and/or lower portion 101A, 101B of the cardiac implant device 101 to the annulus 20 of the valve with the securing member 102.

In addition the medical securing device 100 may also comprise a second elongated stem 107 adjacent to the first stem, as is described in more details in FIG. 12, but also in FIGS. 3, 4, 6, 7, 8. The first 103 and second 107 stems are configured to be moved advantageously under the same manipulation or movement in relation to each other in their longitudinal direction 108. The distal end 107B of the second stem 107 is advantageously configured to be introduced to the opposite portion of the valve tissue than the distal end 103B of the first stem 103 or to the lower portion 101B of the cardiac implant device 101, when the cardiac implant device 101 comprises the lower portion 101B.

The distal end 107B of the second stem 107 has advantageously a counterpart portion 109, which can be used for clamping and thereby controlling the cardiac implant device 101 between the counterpart portion 109 and the distal end 103B of the first stem for further operation, namely for introduction the securing member 102. In addition the counterpart portion 109 can be used, together with the receiving member 104, for securing, such as stapling the securing member 102 to the cardiac implant device 101 and/or to the annulus 20. As can be seen in Figures the counterpart portion 109 can be used as an anvil to guide and bend the securing member 102 around or into the contact with the cardiac implant device 101 and/or to the annulus 20, when introduced by the receiving member 104.

Especially the distal end 107B is configured to produce counterforce via the counterpart portion 109 to the distal end 103B of the first stem 103, for example when the second stem 107 is pulled (107A, 114). For example the operation member illustrated in FIG. 13 can be arranged so that when the proximal end 103A, 107A of the first and/or second stem(s) 103, 107 and/or the operation member 114 are pressed against each other, the distal end of the first and/or second stems is/are moved in the longitudinal direction 108 towards the cardiac implant device 101, and the cardiac implant device 101 is then clamped between the distal ends 103B, 107B of the first and second stems 103, 107. In addition during the additional movement 108 the distal end 103B of the first stem 103 with the receiving member 104 or the receiving member 104 as such is configured to introduce the securing member 102 to the cardiac implant device 101 to secure at least portion of the cardiac implant device 101 to the annulus 20 of the valve with the securing member 102.

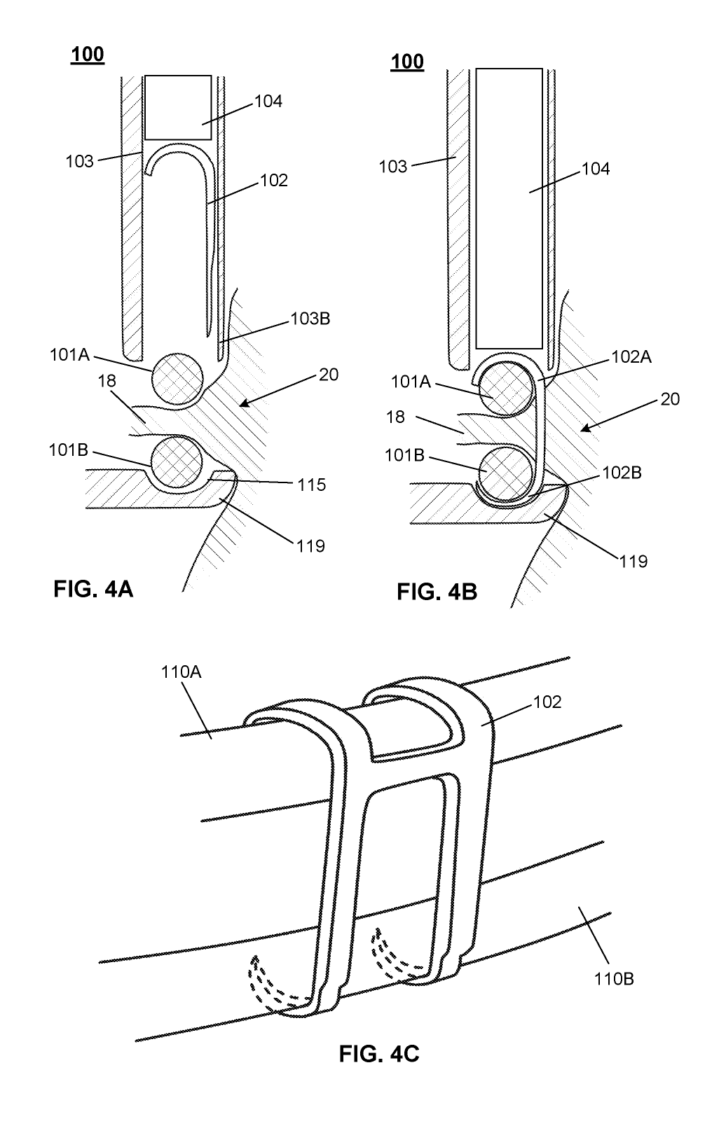

The distal end 103B of the first stem 103 or the receiving member 104 is configured to press and/or bend a first end 102A of the securing member at least partially around or partially into the upper and/or lower portion 101A, 101B of the cardiac implant device 101, when the distal end 103B of the first stem 103 and/or the receiving member 104 is pressed against the counterpart portion 109 of the second stem 107. As can be seen e.g. in FIGS. 3A and 3B the ends 102A, 102B of the securing member 102 are pressed and bended into the slots 117 in the cardiac implant device 101. The securing member 102 may be as a nail or having H-form (see. e.g. FIG. 3C), and the first end 102A of it can have a hook shape originally, whereupon the second end 102B can be bend by the counterpart portion 109 functioning as an anvil. Alternatively the securing member 102 can be introduced around the upper and lower portions 101A, 101B of the cardiac implant device 101, as is illustrated in FIG. 4A-4C. Also in this example the first end 102A of the securing member 102 comprises a hook shape originally, and the second end 102B is formed and guided around the lower portion 101B of the cardiac implant device 101 by the counterpart portion 109 functioning as the anvil.

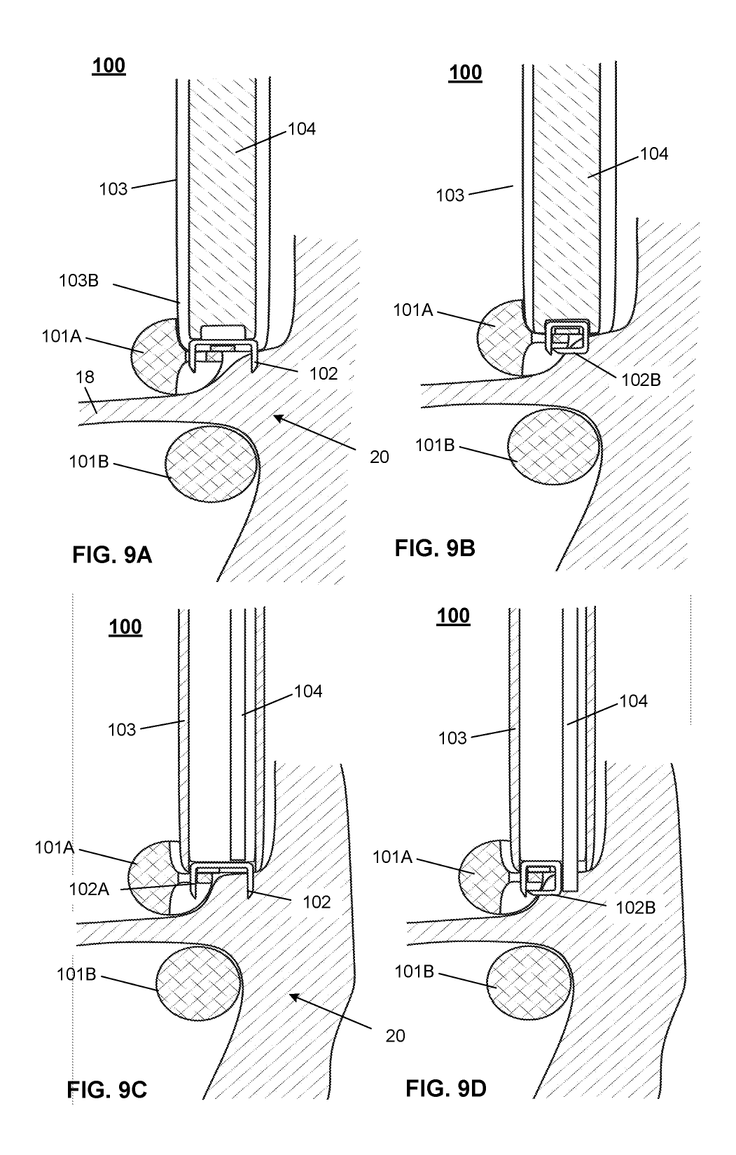

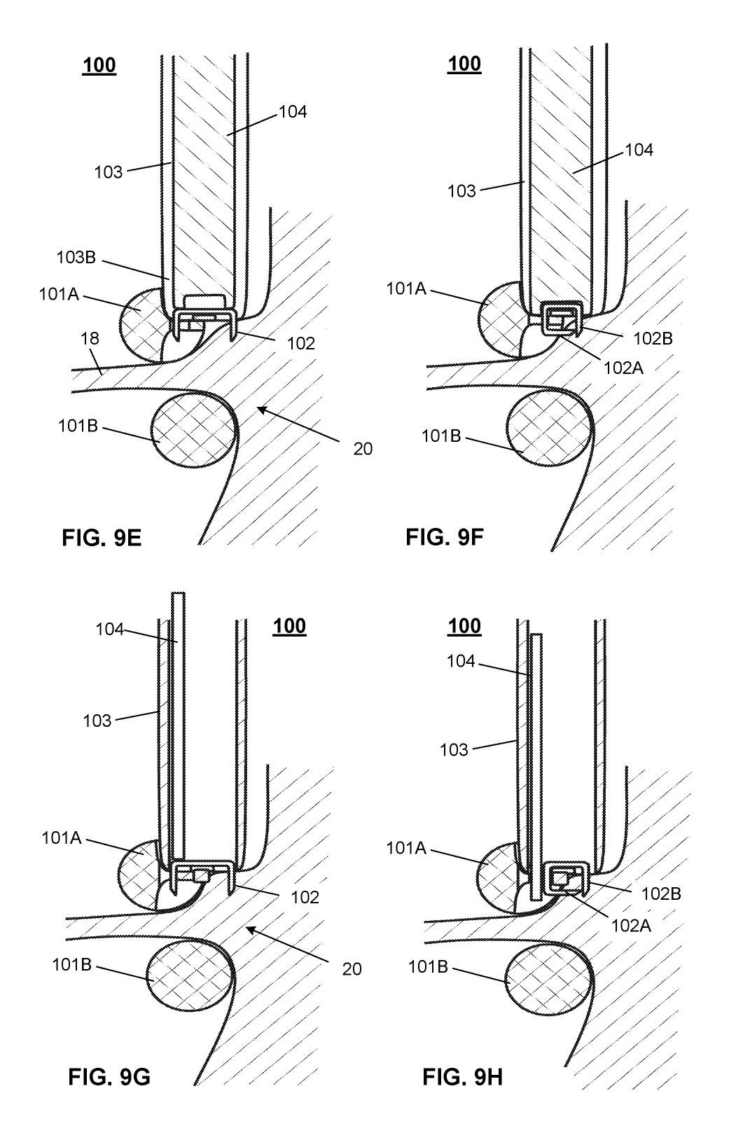

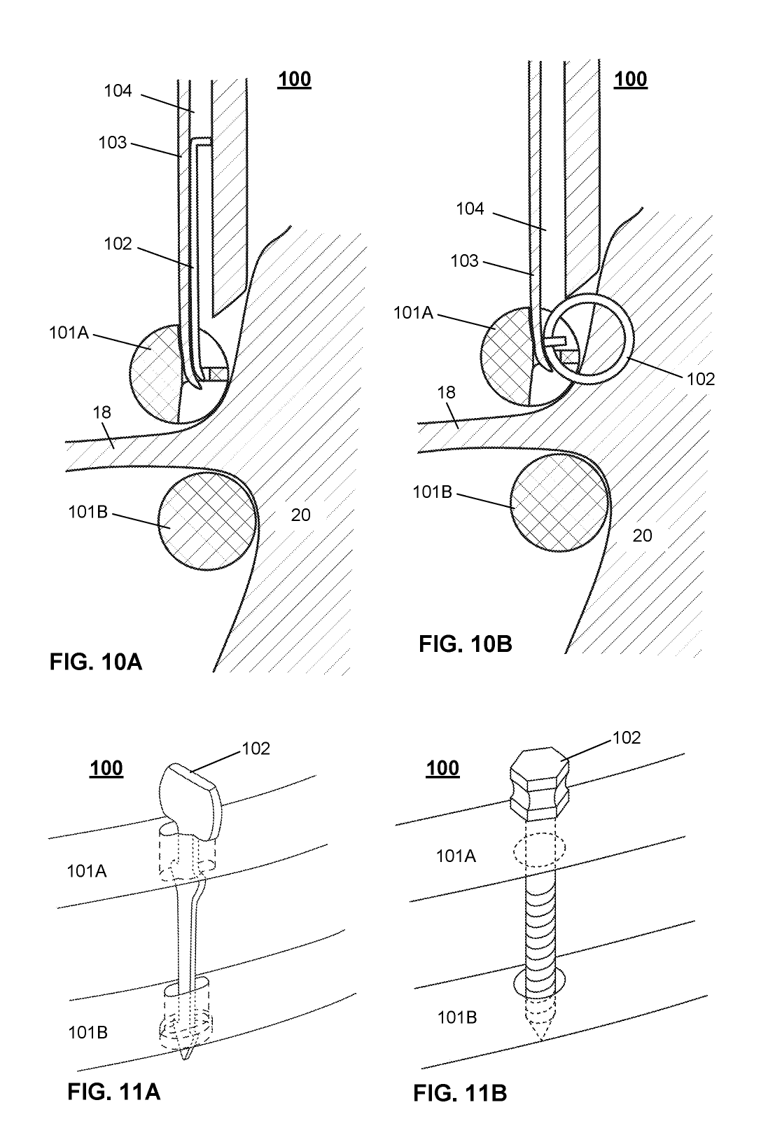

It also should be noted that the distal end 103B of the first stem 103 (advantageously with the receiving member (104)) as such or together with the counterpart portion 109 of the second stem 107 may also be configured to press and/or bend the second end 102B of the securing member 102 at least partially into the annulus 20 of the valve, as is described in FIG. 9A-9D, or FIG. 10A-10B. In particularly in FIG. 9A-9B the receiving member 104 can be arranged so that when it is pulled upwards it will raise the centre portion of the securing member 102 (as a staple), whereupon the ends of the securing member 102 will bend downwards and are thereby introduced into the cardiac implant device 101 and the annulus tissue 20. In FIG. 9C-9D the receiving member 104 (such as a piston type member) is pushed downwards, whereupon it will bend at least one end of the securing member 102 (as a staple) downwards and is thereby introduced into the cardiac implant device 101 and the annulus tissue 20. The securing member 102 may also comprise shape memory material, which additionally helps to achieve the form and securing of the securing member e.g. to the annulus as is described in FIG. 10A-10B.

It is to be noted that in the embodiment illustrated in FIG. 9A-9D the first end 102A of the securing member 102 is introduced into or coupled with the cardiac implant device 101 and the second end 102B of the securing member 102 is introduced into or coupled with the annulus tissue 20 (or bend via the annulus tissue towards or into the cardiac implant device 101). Again it is to be noted that in the embodiment illustrated in FIG. 9E-9H the first end 102A of the securing member 102 is introduced into or coupled with the cardiac implant device 101 and then bend towards and further into the annulus tissue 20, and the second end 102B of the securing member 102 is introduced directly to the annulus tissue 20.

As can be seen in the embodiment and drawings, the present invention offer many advantageously features and ways to secure the cardiac implant device into the annulus. For example, the embodiment illustrated in FIG. 9E-9H offer clear advantage, namely at first the introduction of the securing member 102 as well as the securing of the cardiac implant 101 can be done by the medical securing device 100 having only the first stem 103 (either “upper” 103 or “lower” 107 stem. In this embodiment the distal end 103B of the first stem 103 (or 107) with the receiving member (104) is configured to introduce the securing device 102 at least partially through or via the cardiac implant device 101 into the annulus 20 of the valve during the movement of the distal end 103B of the first stem 103 (or 107) and/or the receiving member 104 in relation to the cardiac implant device 101. The movement can be e.g. downwards so towards the cardiac implant device 101 or upwards so away from the cardiac implant device 101.

However when the first end 102A of the securing member is 102 is introduced into or coupled with the cardiac implant device 101 and then bend towards and further into the annulus tissue 20 (and the second end 102B of the securing member 102 is introduced “directly” to the the annulus tissue 20), huge advantage can be achieved, namely when the end (102A) of the securing member 102 to be introduced into the annulus tissue 20 is bend before introduction, the tensile stress, as well as any other further tissue tearing caused to the tissue can be remarkably reduced.

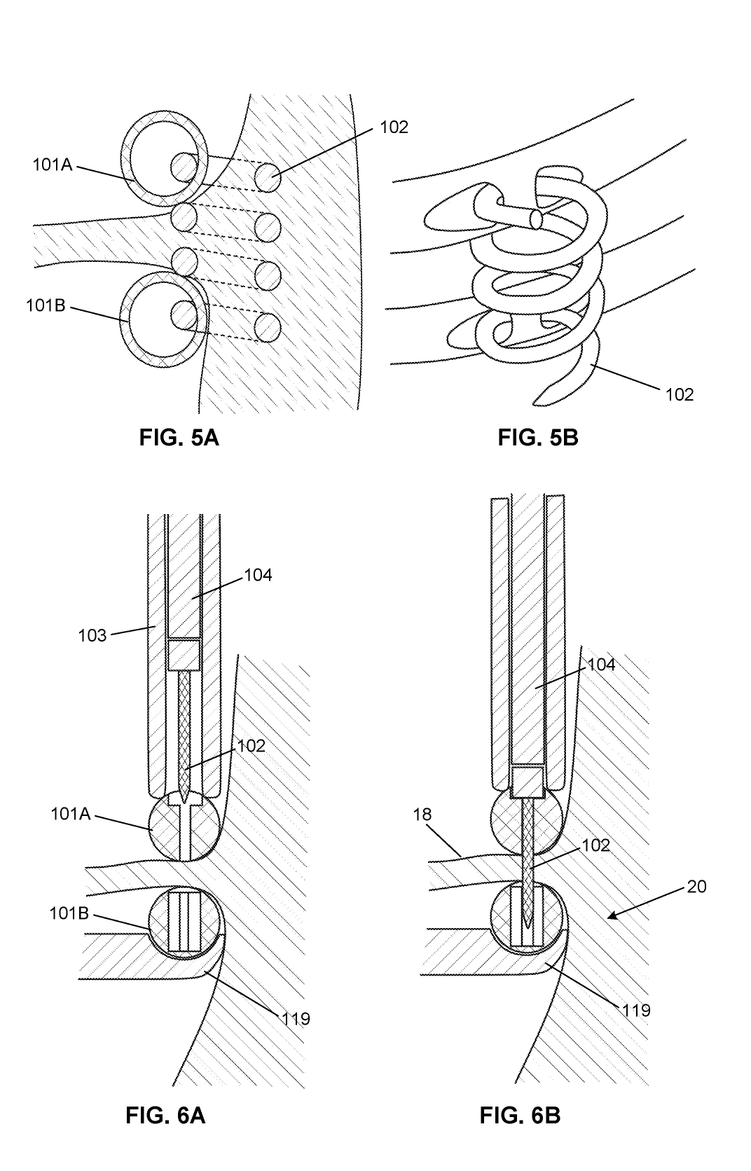

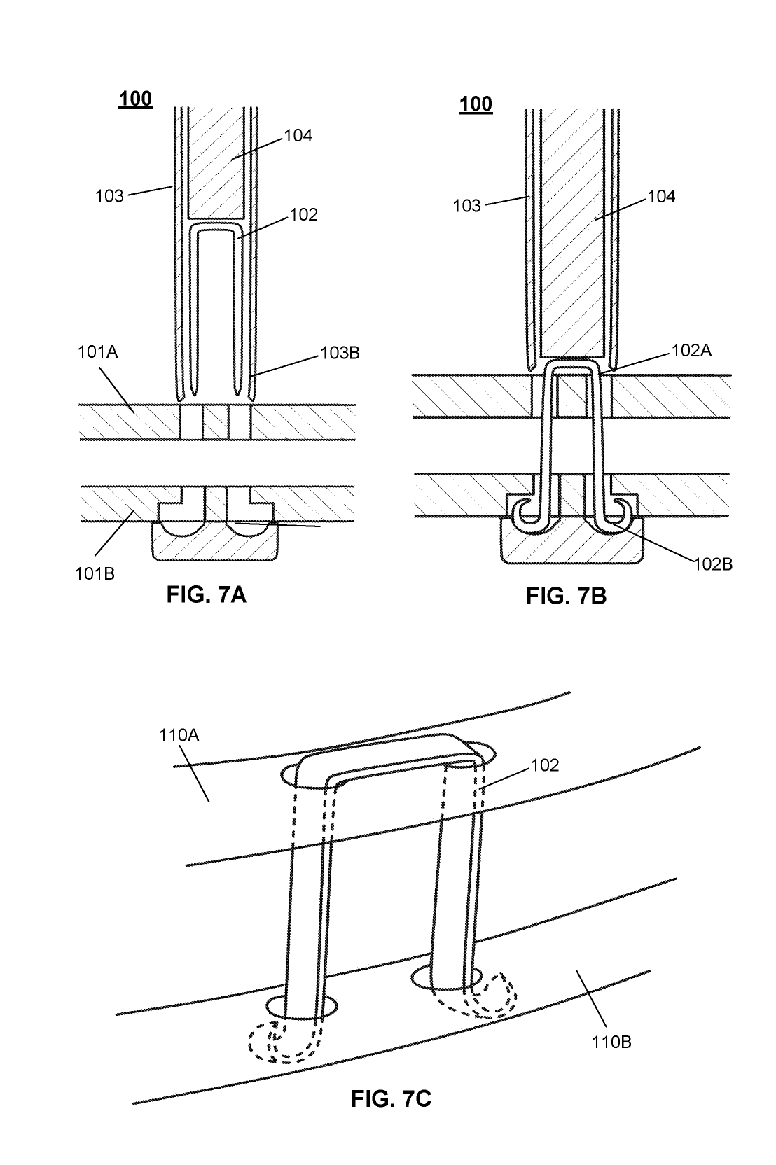

In addition it is to be noted that the securing member 102 may be as a locking clip or pointed screw or the like as is described in FIG. 11A, 11B, whereupon the receiving member 104 can be turned e.g. via manipulation of the operation member 114 so to introduce and turn (screw) the securing member 102 into the cardiac implant device 101. Moreover, as can be seen in FIG. 5A, 5B, the securing member 102 may be as a helical spring 102, whereupon the receiving member 104 possibly together the distal end 103B of the first stem 103 is configured to introduce the helical spring 102 so to penetrate a possible hollow or hole structure in the cardiac implant device 101 as well as to penetrate into the annulus 20 tissue of the valve structure. The cardiac implant device 101 may also comprise a textile structure around it to which the helical spring 102 can be secured. Still in FIG. 6A, 6B is shown a locking clip 102, which can be just pushed through the first hole in the upper portion of the cardiac implant device 101 and again into the receiving securing hole in the lower portion of the cardiac implant device 101, where the securing hole locks the locking clip 102 in its securing position. Additionally FIG. 7A-7C illustrates a hook shaped securing member 102 which is pressed by the receiving member 104 thought the holes in the upper portion 101A of the cardiac implant device 101 and again so that the second end 102B of the hook shaped securing member 102 is bend into the slots of the lower portion 101B of the cardiac implant device 101.

The distal end 103B of the first stem 103 may also comprise a recess 116 for supporting the distal end 103B of the first stem 103 to the upper portion 101A of the cardiac implant device 101. The distal end 107B of the second stem 107 may also comprise a recess 117 for supporting the distal end 107B of the second stem 107 to the lower portion 101B of the cardiac implant device 101 during the introduction of the securing member 102. This helps and guides the securing device 100 to be positioned in an appropriate position for clamping as well as for introducing the securing member and thereby for securing the cardiac implant device 101 into the annulus.

As can be seen especially in FIG. 12 the mutual design of the distal end portions 103B, 107B of the first and second stems 103, 107 are so that a space 110 is formed between the distal end portions. As an example, the space 111 the second stem 107 may comprise a protrusion portion 111 in the distal end portion 107B 110 between the first and second stems 103, 107. In a use the space 110 is configured to receive at least portion of the valve tissue and/or leaflets 18. In addition the distal end 107B of the second stem may have an angle 112, such as inclined or rectangular portion, which extends towards the extension line 113 of the distal end 103B of the first stem 103 so that the counterpart portion 109 of the second stem 107 locates essentially in the extension line 113 in the longitudinal direction 108 of the first stem distal end portion 103B.

The distal end 103B of the first stem 103 has a hollow structure 106, such as a conduit or channel, where the receiving member 104 is arranged in a movable manner. In addition the first stem 103 has a hollow structure 106 via which the operation member 114 is functionally coupled with the receiving member 104. During the movement the receiving member 104 moves and when it is moved towards the output of the distal end 103B, it thereby introduces the securing member from the hollow structure to the cardiac implant device 101 and secures the cardiac implant device (or at least part of it) to the annulus 20 of the valve with the securing member.

The medical securing device may also comprise a storage (not shown in Figures) for receiving number of securing members 102. The storage is advantageously arranged to feed a new securing member 102 when the previous one is introduced to the cardiac implant device 101.

The receiving member 104 can be a piston type, for example, such as is described e.g. in FIGS. 6A, 6B, 7A, 7B, 8A, 8B, 9A-9H. The piston type receiving member 104 can be operated e.g. by operating, such as pulling an operation member 114, which is advantageously comprised by or in the proximal end 103A of the first stem 103. The operation member 114 is advantageously configured for moving the distal end 103B of the first stem 103, or especially the receiving member 104 towards the cardiac implant device 101 and thereby introducing the securing member 102 to the cardiac implant device 101. Most advantageously the operation member 114 moves the piston type receiving member 104 so that the securing device 102 is outputted at least partially from the distal end 103B or from the hollow structure 106 and against the cardiac implant device 101, after which and advantageously during the same movement the second stem 107 is pulled thereby clamping the cardiac implant device 101 and after this introducing the securing device 102 further and around or into the contact with cardiac implant device 101 and thereby securing said cardiac implant device 101 into the annulus 20.

The operation member 114 advantageously together with the first and/or second stems 103, 107 is/are arranged so that during an operation the following operations are configured to happen either simultaneously or in sequentially by the same operation or movement of the operation member 114:
the movement of the distal ends 103B, 107B of the first and second stems 103, 107 in relation to each other and towards to each other (pull e.g. the second stem) in their longitudinal direction 108 and thereby to clamp cardiac implant device 101 between the distal ends 103B, 107B of the first and second stems 103, 107,
the movement of the distal end 103B of the first stem 103, and/or the movement of the receiving member 104 towards the cardiac implant device 101,
positioning the cardiac implant device 101 in the securing position so i.e. pulling or pushing the cardiac implant device 101 or its lower or upper portion around or over the annulus 20 in its proper securing position, and/or
introduction the securing member 102 to the cardiac implant device 101 to secure the cardiac implant device 101 to the annulus 20 of the valve with the securing member 102.

As can be seen in Figures the cardiac implant device 101 comprises the upper and/or lower portion 101A, 101B, which are advantageously loop-shaped portions. They may form a coil-shape portion so that during an insertion the cardiac implant device 101 can be rotated into the position. In an advantageous embodiment at least one of the portions 101A, 101B is an open shaped, such as a C or D shaped portion.

The invention has been explained above with reference to the aforementioned embodiments, and several advantages of the invention have been demonstrated. It is clear that the invention is not only restricted to these embodiments, but comprises all possible embodiments within the spirit and scope of the inventive thought and the following patent claims.

For example when it is stated that it is the first elongated stem having proximal, which is used to introduce the securing member to the cardiac implant device to secure the cardiac implant device to the annulus of the valve with the securing member, it should be understood that this first stem (if no other stem is mentioned or needed) can be functionally either the “upper” stem (103) or the “lower” stem (107), by which the introduction and securing can be performed. In addition it is to be noted that the medical securing device according to the invention can be used in trancatheter applications, such as for example trancatheter mitral valve implantation. In particularly the first and second elongated stems can be used together with a steering catheter, which is big enough.

The features recited in dependent claims are mutually freely combinable unless otherwise explicitly stated.

1. A medical securing device for securing an implant device with a securing member into a tissue, where the implant device comprises a upper or lower portion, whereby, in use, a portion of the tissue is coupled with the upper or lower portion, or trapped between the upper and lower portions of the implant device, wherein the medical securing device comprises:
a first elongated stem having proximal and distal ends,
where
the distal end of the first stem has a receiving member for receiving the securing member,
whereupon during a movement of the distal end of the first stem and the receiving member in relation to each other, the distal end of the first stem with the receiving member is configured to introduce the securing member to the implant device to secure the implant device to the tissue with the securing member.

2. The medical securing device of claim 1, wherein the distal end of the first stem has a hollow structure, and where the receiving member is arranged to the distal end in a movable manner in relation to the cavity so that during the movement the receiving member is configured to introduce the securing member from the cavity of the distal end to the implant device to secure the implant device to the tissue with the securing member.

3. The medical securing device of claim 1, wherein the medical securing device further comprises:
a second elongated stem adjacent to the first stem, where said first and second stems are configured to be moved in relation to each other in their longitudinal direction, both stems having proximal and distal ends, where
the distal end of the second stem has a counterpart portion for the distal end of the first stem or for the receiving member of the first stem,
the distal end of the second stem is configured to be introduced to the opposite portion of the tissue than the distal end of the first stem and to produce counterforce via the counterpart portion to the distal end of the first stem and thereby clamping the implant device between the distal ends of the first and second stems when the distal end of the first or second stems is/are moved in the longitudinal direction towards the implant device, and wherein in addition during the movement the distal end of the first stem with the receiving member is configured to introduce the securing member to the implant device to secure the implant device to the tissue with the securing member.

4. The medical securing device of claim 3, wherein the mutual design of the distal end portions of the first and second stems are so that a space is formed between the distal end portions of the first and second stems, whereupon, in use, the space is configured to receive at least portion of the tissue.

5. The medical securing device of claim 3, wherein the second stem has a protrusion portion in the distal end portion forming the space between the first and second stems.

6. The medical securing device of claim 3, wherein the distal end of the second stem has an angle, towards the extension line of the distal end of the first stem so that the counterpart portion of the second stem locates essentially in the extension line in the longitudinal direction of the first stem distal end portion.

7. The medical securing device of claim 2, wherein the proximal end of the first stem comprises an operation member for moving the distal end of the first stem, or the receiving member towards the cardiac implant device and thereby introducing the securing member to the cardiac implant device.

8. The medical securing device of claim 3, wherein the proximal end of the first stem comprises an operation member during an operation of which the following operations are configured to happen either simultaneously or in sequentially by the same operation of the operation member:
the movement of the distal ends of the first and second stems in relation to each other and towards to each other in their longitudinal direction and thereby to clamp implant device between the distal ends of the first and second stems,
the movement of the distal end of the first stem, or the movement of the receiving member towards the implant device,
positioning the implant device in the securing position, or
introduction of the securing member to the implant device to secure the implant device to the tissue with the securing member.

9. The medical securing device of claim 7, wherein the first stem has a conduit via which the operation member is functionally coupled with the receiving member.

10. The medical securing device of claim 3, wherein the distal end of the first stem or the receiving member is configured to press or bend a first end of the securing member at least partially around or partially into the upper or lower portion of the implant device, when the distal end of the first stem is pressed against the counterpart portion of the second stem.

11. The medical securing device of claim 1, wherein the distal end of the first stem or the counterpart portion of the second stem configured to press or bend a second end of the securing member at least partially around or partially into the lower portion of the implant device or into the tissue, when the distal end of the first stem is pressed against the counterpart portion of the second stem.

12. The medical securing device of claim 3, wherein the counterpart portion comprises an anvil for receiving and bending the securing device at least partially around or partially into the implant device or to the tissue during the movement of the distal end of the first stem towards the implant device.

13. The medical securing device of claim 1, wherein the distal end of the first stem with the receiving member is configured to introduce the securing device at least partially through or via the implant device into the tissue during the movement of the distal end of the first stem or the receiving member in relation the implant device.

14. The medical securing device of claim 1, wherein the distal end of the first stem comprises a recess for supporting the distal end of the first stem to the upper portion of the implant device or the distal end of the second stem comprises a recess for supporting the distal end of the second stem to the lower portion of the implant device during the introduction of the securing member.

15. The medical securing device of claim 1, wherein the medical securing device comprises a storage for receiving a number of securing members, whereupon the medical securing device is arranged to feed a new securing member from the storage when the previous one is introduced to the implant device.

16. The medical securing device of claim 1, wherein the medical securing device is a cardiac implant device for an open-heart-operation, and the tissue is an annulus of the heart valve.

17. The medical securing device of claim 1, wherein the securing member is one of the following:
a staple, the one end of which is bendable at least partially around or into the cardiac implant device under the pressing force induced by the distal end of the first stem or counterpart portion of the second stem,
a staple having at least one hook-shaped end to be introduced at least partially around or into the cardiac implant device,
a helical clip,
a locking clip,
a pointed screw,
a spring clip,
a skin staple, or
a circular clip.

18. The medical securing device of claim 1, wherein the securing member comprises shape memory material, metal or polymer.

19. The medical securing device of claim 1, wherein the implant device comprises the upper or lower portion, which are advantageously loop-shaped portions arranged to form a coil-shape so that during an insertion the implant device is rotated into the position, and wherein at least one of the portion is at least partially open shaped.

20. The medical securing device of claim 3, wherein the first elongated stem is made of one piece of material, and wherein the second elongated stem is made of one piece of material.
